(19) United States
(12) Patent Application Publication
(10) Pub. No.: US 2026/0240500 A1
(43) Pub. Date: Aug. 20, 2026

(54) REPOSITIONABLE VOLAR MODULE

(71) Applicant: ROCKLEY PHOTONICS LIMITED, Altrincham (GB)

(72) Inventors: Kate LeeAnn BECHTEL, Pleasant Hill, CA (US); Cody DUNN, Costa Mesa, CA (US); James McMILLAN, Santa Monica, CA (US)

(21) Appl. No.: 19/643,096

(22) Filed: Apr. 9, 2026

Related U.S. Application Data
(63) Continuation of application No. 18/480,492, filed on Oct. 3, 2023, now abandoned.
(60) Provisional application No. 63/413,181, filed on Oct. 4, 2022.

(51) Int. Cl. A61B 5/00

(52) U.S. Cl. CPC A61B 5/7221; A61B 5/7235

(57) ABSTRACT

A wearable volar module includes a biometric sensor configured to produce a photoplethysmography signal and a speckleplethysmography signal, the volar module being configured to be attached to a wearable strap, and the wearable strap being configured to be attached to a dorsal module.

### CROSS-REFERENCE TO RELATED APPLICATION(S)

This application is a continuation of U.S. Patent Application No. 18/480,492 filed October 3, 2023, which claims priority to and the benefit of U.S. Provisional Application No. 63/413,181, filed October 4, 2022, entitled "REPOSITIONABLE VOLAR MODULE", the entire contents of which are incorporated herein by reference.

### FIELD

One or more aspects of embodiments according to the present disclosure relate to biometric monitoring, and more particularly to a repositionable volar module.

### BACKGROUND

A biometric monitoring system may be worn on the wrist of a subject, and may perform various biometric measurements on the dorsal side of the wrist. Some biometric measurements, e.g., ones which are based on measurements of arterial blood, may be performed on the volar side of the wrist.

It is with respect to this general technical environment that aspects of the present disclosure are related.

### SUMMARY

According to an embodiment of the present disclosure, there is provided a system, including: a first wearable instrument; a second wearable instrument including a biometric sensor; a conductive connection between the first wearable instrument and the second wearable instrument; and a strap, sized and dimensioned to be disposed about a wrist, the system being capable of: securing the first wearable instrument to the strap; securing the second wearable instrument to the strap at a first position relative to the first wearable instrument; and securing the second wearable instrument to the strap at a second position relative to the first wearable instrument.

In some embodiments, the conductive connection is configured to supply power, from the first wearable instrument to the second wearable instrument.

In some embodiments, the first wearable instrument includes a battery configured to supply power to the first wearable instrument and the second wearable instrument.

In some embodiments, the conductive connection is configured to transmit signals from the second wearable instrument to the first wearable instrument.

In some embodiments, the first wearable instrument includes a radio configured to transmit measurement data.

In some embodiments, the first wearable instrument is configured: to receive measurement data obtained by the second wearable instrument; and to transmit the measurement data, via the radio.

In some embodiments, the conductive connection is further configured to transmit signals from the first wearable instrument to the second wearable instrument.

In some embodiments: in the first position, the second wearable instrument is separated from the first wearable instrument by a first distance along the strap; in the second position, the second wearable instrument is separated from the first wearable instrument by a second distance along the strap; and the first distance differs from the second distance by less than 5 mm.

In some embodiments, the first distance differs from the second distance by less than 2 mm.

10. The system of claim 1, wherein the system is capable of: securing the second wearable instrument to the strap at a plurality of positions, including the first position and the second position, relative to the first wearable instrument; and the plurality of positions includes 5 distinct positions.

In some embodiments, the plurality of positions includes 20 distinct positions over a range of distances, along the strap, between the first wearable instrument and the second wearable instrument, the range of distances including: a first distance, and a second distance greater than the first distance by 5 mm.

In some embodiments, the first wearable instrument includes a spectrophotometer.

In some embodiments, the biometric sensor is a sensor selected from the group consisting of photoplethysmography sensors, speckleplethysmography sensors, speckle imaging sensors, and diffuse correlation spectroscopy sensors.

In some embodiments, the system further includes a third wearable instrument, wherein the system is further capable of securing the third wearable instrument to the strap.

In some embodiments, the system includes one or more processing circuits configured: to receive a sequence of measurements from the second wearable instrument; and to calculate a signal quality indicator.

In some embodiments, the one or more processing circuits are further configured to display the signal quality indicator to a user.

In some embodiments, the signal quality indicator is based on a frequency-domain analysis of the sequence of measurements.

In some embodiments, the signal quality indicator is based on a plurality of fiducial points in a waveform corresponding to the sequence of measurements.

In some embodiments, the signal quality indicator is based on a morphological analysis of a waveform corresponding to the sequence of measurements.

### BRIEF DESCRIPTION OF THE DRAWINGS

These and other features and advantages of the present disclosure will be appreciated and understood with reference to the specification, claims, and appended drawings wherein:

FIG. 1A is a side view of a system for monitoring biometrics, according to an embodiment of the present disclosure;

FIG. 1B is a side view of a system for monitoring biometrics, according to an embodiment of the present disclosure;

FIG. 1C is a block diagram of a spectrophotometer, according to an embodiment of the present disclosure;

FIG. 2A is a flow chart of a method for adjusting the position of a volar module; and

FIG. 2B is a table showing display methods for displaying a signal quality indicator.

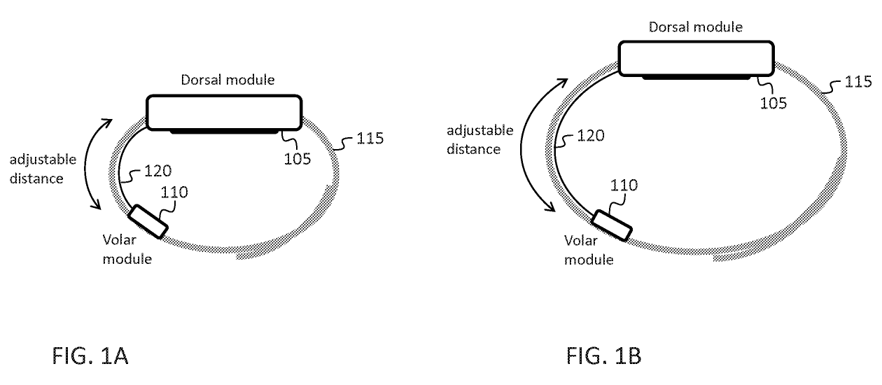

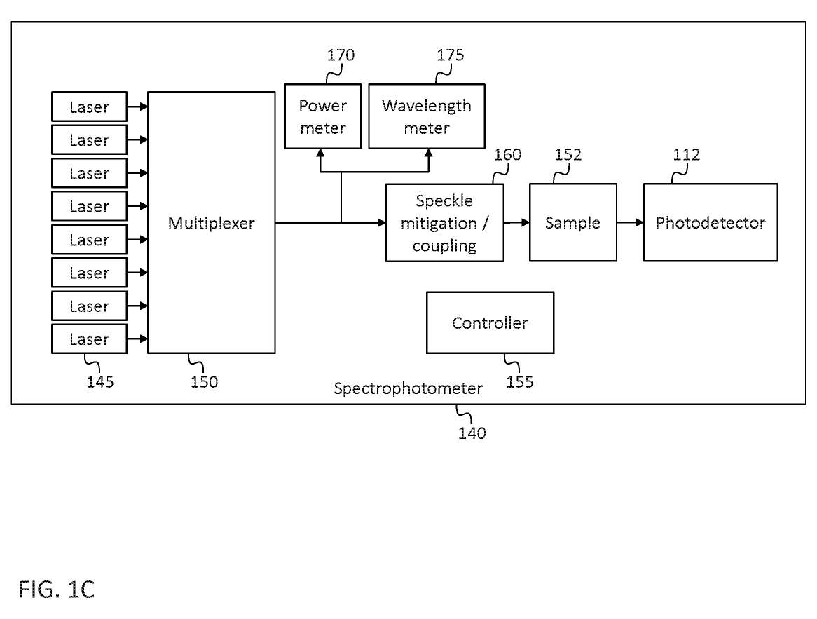

Each of FIGS. 1A and 1B is drawn to scale, for one respective embodiment.

### DETAILED DESCRIPTION

The detailed description set forth below in connection with the appended drawings is intended as a description of exemplary embodiments of a repositionable volar module provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

FIG. 1A shows a system for monitoring biometrics of a subject (e.g., of a patient). As used herein, “biometrics” are physiological parameters, examples of which are given below. The subject may wear the system of FIG. 1A on her or his wrist. The system may include, as illustrated, a first wearable instrument 105 and a second wearable instrument 110, both of which may be secured, in use, to the subject’s wrist by a strap (e.g., a flexible strap) 115. In some embodiments, the first wearable instrument 105 is larger than the second wearable instrument 110. The subject may be accustomed to wearing (and may prefer to wear) a wrist-worn device on the dorsal side (the top side) (e.g., in the dorsal central zone) of the wrist. As such, one of the wearable instruments may be worn on the dorsal side of the wrist, and, in embodiments in which one of the wearable instruments is larger than the other, the larger one of the wearable instruments (e.g., the first wearable instrument 105) may be worn on the dorsal side of the wrist. In such a configuration, various biometrics may be measured by the first wearable instrument 105. Some biometrics, however, are more readily measured or are only possible to be measured on the volar side (palm side) of the wrist where more direct access to arterial blood flow can be achieved.

A standalone wrist-worn wearable instrument that measures on the volar side of the wrist may be too bulky (e.g., because of the presence of inclusion of optics, electronics, or other sensor components) to be comfortably worn on the volar side of the wrist and may cause discomfort when operating a computer keyboard or mouse or any time the subject is in a wrist prone position. In some embodiments, the width of the wearable instrument may be large to cover the possible variations in anatomy such that the region of interest, for example the radial or ulnar artery, is at least partially covered. In a system for monitoring biometrics in which a single, rigid, curved housing includes two wearable instruments (a first one intended to perform measurements on the dorsal side of the wrist, and a second intended to perform measurements on the volar side of the wrist), the comfort of the subject may be affected by the bulk and rigidity of the housing, and, if the system fits well on a subject with a large wrist, the second (volar) wearable instrument may be poorly positioned on a subject with a small wrist, or vice versa. Even in a system including two wearable instruments on a flexible support (e.g., a strap or band), if the position of the second wearable instrument relative to the first wearable instrument is fixed, then the positioning of the second wearable instrument, when the first wearable instrument is centered on the dorsal side of the wrist, may be poor for at least some subjects, compromising signal quality.

As such, some embodiments include a first wearable instrument 105 and a second wearable instrument 110 (e.g., as illustrated in FIG. 1A), and the second wearable instrument 110 is independently repositionable with millimeter-scale resolution over a relatively large distance with respect to the first wearable instrument 105. For example, the system illustrated in FIG. 1A may be capable of securing the second wearable instrument 110 at any one of a plurality of positions (including, e.g., a first position, and a second position) along the strap 115, relative to the first wearable instrument 105. This adjustability may be made possible by adjustability of the position of the first wearable instrument 105 along the strap 115, or by adjustability of the position of the second wearable instrument 110 along the strap 115, or both. Due to variations in human wrist circumference (which may vary, e.g., from 136 mm to 193 mm), the distance between the first wearable instrument 105 and the second wearable instrument 110, when the two wearable instruments are in respective optimal positions, may vary as much as 38 millimeters (mm) from a subject with a large wrist to a subject with a small wrist. The strap 115 may therefore be tightened or loosened (e.g., from 136 mm to 193 mm) to adjust to the size of the wrist of the subject, and the position of the second wearable instrument 110 relative to the first wearable instrument 105 on the strap 115 may be adjusted (e.g., independently adjusted, through a range of positions spanning 38 mm) so that the second wearable instrument 110 is at a position on the wrist at which acceptable signal quality may be obtained by one or more biometric sensors in the second wearable instrument 110. Such positions may be over a first relatively narrow region directly over the radial artery (or over a second relatively narrow region directly over the ulnar artery), and, as such, it may advantageous for the position of the second wearable instrument 110, relative to the position of the first wearable instrument 105, to be adjustable in relatively fine increments (e.g., increments of between 0.1 mm and 5.0 mm).

FIG. 1B shows the embodiment of FIG. 1A, with the circumference of the strap 115 adjusted (e.g., loosened) to accommodate a larger wrist than that of FIG. 1A, and with the position of the second wearable instrument 110 adjusted on the strap 115 so that the second wearable instrument 110 is more distant from the first wearable instrument 105 than it is in the configuration of FIG. 1A. FIGS. 1A and 1B shows an electrical connection 120 (discussed in further detail below) between the first wearable instrument 105 and the second wearable instrument 110. As used herein, the “circumference of the strap” when the strap 115 is adjusted to a certain size means the inner circumference of the strap 115 (when the strap 115 is closed, if it is a strap 115 that is capable of being opened and closed), e.g., the circumference of the strap is the circumference of the largest wrist the strap 115 will accommodate when so adjusted.

In some embodiments the system includes a third wearable instrument, which may be secured to the strap 115 at a third position on the wrist. For example, the second wearable instrument 110 may be adjusted to be positioned over the radial artery and the third wearable instrument may be adjusted to be positioned over the ulnar artery. In such an embodiment an electrical connection between the first wearable instrument 105 and the third wearable instrument or an electrical connection between the second wearable instrument 110 and the third wearable instrument may make it possible to supply power to, and receive data from, the third wearable instrument (as discussed in further detail below, for the second wearable instrument 110). In some embodiments, one or more of the wearable instruments does not include a biometric sensor. In such an embodiment, such a wearable instrument may, e.g., include a battery or a processing circuit (and no biometric sensors), and one or more biometric sensors may be present in one or more of the other wearable instruments (or in the other wearable instrument, if there is only one other wearable instrument).

The size of the strap 115, the position of the first wearable instrument 105 on the strap 115, and the position of the second wearable instrument 110 on the strap 115 may each be held (e.g., prevented from varying, after having been adjusted) by any of various kinds of clasps. As used herein, a “clasp” is a fitting that secures something to a location along the length of the strap 115. As such, a buckle, secured to a first end of the strap 115 (e.g., by a bar extending through a loop in the first end of the strap 115), with a prong that may be inserted into any of a plurality of holes in the strap 115, near a second end of the strap 115, may be a clasp. Similarly, each of a ladder lock, a cinch lock, a cam buckle, a tri glide, a tri glide slide buckle, and a hook and loop (e.g., Velcro^{TM}) fastener (which may be used with a buckle) is an example of a clasp. In some embodiments, a first clasp is used to set the circumference of the strap 115, a second clasp is used to set the position of the first wearable instrument 105 on the strap 115 and a third clasp is used to set the position of the second wearable instrument 110 on the strap 115.

The position of a wearable instrument may be set by a clasp e.g., by a clasp secured directly to the wearable instrument, or, in an embodiment in which the wearable instrument is secured to a portion of the strap 115, by a clasp setting the position of the portion of the strap 115 relative to the remainder of the strap 115. The strap 115 may be a single (e.g., fabric or elastomer) strip or it may include more than one strip of, e.g., fabric or elastomer. For example, it may include two pieces, connected to the first wearable instrument 105 in a manner similar to that of a two-piece watch band; it may include a first portion connected to a first bar on the first wearable instrument 105 (e.g., by looping around the first bar) and a second portion connected to a second bar on the first wearable instrument 105 (e.g., by looping around the second bar). In such an embodiment the free and of the first portion (the end not secured to a bar on the first wearable instrument 105) may, like a corresponding end of a watch band, be secured to the free end of the second portion in an adjustable manner (e.g., using a buckle on the end of the first portion with a prong that may be inserted into any one of a series of holes in the second portion) to accommodate various wrist sizes. In some embodiments, a one-piece strap may pass through each of two gaps between the housing of the first wearable instrument 105 and respective bars secured to the housing. At each such gap the strap 115 may change direction, and the bars and the pair of gaps may operate as a clasp securing the first wearable instrument 105 to the strap 115 by friction.

The electrical connection 120 may include a conductive connection including one or more (e.g., two or more) conductors, e.g., for transmitting power or signals between the first wearable instrument 105 and the second wearable instrument 110. The conductive connection may include dedicated conductors or shared conductors (e.g., it may include a first power conductor, a first data conductor, and a shared ground conductor, the shared ground conductor being used both to form a power connection and a data connection). The electrical connection 120 may be employed for various functions. For example, the first wearable instrument 105 may include a battery supplying power both to (i) circuitry and one or more sensors in the first wearable instrument 105 and to (ii) circuitry and one or more sensors in the second wearable instrument 110. As another example, the first wearable instrument 105 may include a radio and an antenna for conducting wireless communications (e.g., Bluetooth^{TM} or Wi-Fi^{TM} communications) with stationary equipment (e.g., with a piece of medical equipment in a clinic or with a Wi-Fi router) or with a mobile device (e.g., a mobile telephone, laptop computer, or tablet computer), and the first wearable instrument 105 may relay signals (e.g., commands or data) to and from the second wearable instrument 110. The subject or a clinician may use such a mobile device for recording or relaying measurements obtained by the system (e.g., measurement data obtained by the first wearable instrument 105, or measurement data obtained by the second wearable instrument 110 and relayed by the radio of the first wearable instrument 105). In such embodiments, it may be unnecessary for the second wearable instrument 110 to include, for example, a battery, a radio, or an antenna, making it possible for the second wearable instrument 110 to be smaller than it otherwise would be. Other functions (e.g., a real-time clock, storage of subject-specific data, or encryption and decryption of data) may also be provided, for the second wearable instrument 110, by the first wearable instrument 105, to make further size reductions of the second wearable instrument 110 possible.

The volar measurement location of the second wearable instrument 110 may offer the opportunity to measure from positions over, e.g., radial or ulnar arterial flow. The terms “volar”, “radial”, and “ulnar” sensing location all refer to a portion of the wrist that is not the (relatively flat) dorsal side of the wrist. As such, the volar side is not limited to any specific physiological position beyond its relation to the dorsal side of the wrist, and the volar side includes, for example, the palm side of the wrist. In an embodiment in which the first wearable instrument 105 and the second wearable instrument 110 contain the same sensor or sensors it may be possible to allow the user to select the orientation of the wearable instruments with respect to the wrist. In such a case the wearable instruments may be interchangeable from a sensing perspective. In some embodiments, measurements between the first wearable instrument 105 and the second wearable instrument 110 may be compared. In some embodiments, a system such as that of FIG. 1A may be worn at another location other than on the wrist, e.g., on an ankle.

Each of the first wearable instrument 105 and the second wearable instrument 110 may include one or more sensors selected from a variety of sensors suitable for inclusion in a wearable instrument. The measured signals may be optical, including signals measured with either coherent or incoherent light, including photoplethysmography, speckleplethysmography, speckle imaging, diffuse correlation spectroscopy, visible or infrared absorption spectroscopy, fluorescence spectroscopy, radio frequency (RF); acoustic sensing; electrical sensing including bioimpedance and electrocardiography; and pressure sensing including applanation tonometry.

In some embodiments, a wearable instrument may include a spectrophotometer 140 (FIG. 1C), which may be used to measure various biomarkers (which are examples of biometrics that the wearable instruments may measure), each of which may be the concentration of a constituent of the tissues of the subject. FIG. 1C is a block diagram of a spectrophotometer 140, in some embodiments. Each laser 145 of an array of lasers 145 (e.g., ten or more lasers 145, not all of which are shown) is connected to a wavelength multiplexer 150 (which may be, e.g., an arrayed waveguide grating, an echelle grating, or a cascade of Mach-Zehnder interferometers). Each laser 145 may include an InP reflective semiconductor optical amplifier (RSOA) coupled to a waveguide on a silicon photonic integrated circuit (a silicon PIC). The waveguide on the silicon photonic integrated circuit may include a grating reflector that sets the operating wavelength of the laser. Each laser 145 operates at a different respective wavelength and is connected to an input, corresponding to the operating wavelength of the laser, of the wavelength multiplexer 150. In operation, one laser is turned on at a time (e.g., by a controller 155, which may be or include a processing circuit), so that the combination of (i) the array of lasers 145 and (ii) the wavelength multiplexer 150 operates as a swept wavelength light source. In other embodiments, a different swept wavelength light source (e.g., a single widely tunable laser, or a source including an array of tunable lasers, each tunable over a different wavelength range) is used instead of the array of lasers 145 and the wavelength multiplexer 150 shown in FIG. 1C. In the embodiment of FIG. 1C, the wavelength separation between lasers 145 that are adjacent in wavelength may be between 5 nm and 50 nm, and the wavelength range may be about 2000 nm to 2500 nm (e.g., 2080 nm to 2400 nm). In some embodiments, one or more gaps may be present in the set of wavelengths (e.g., if a wavelength band within the range is of limited use because of strong absorption by water in the band).

Light from the output of the wavelength multiplexer 150 illuminates the sample 152. In some embodiments, a speckle mitigation system or coupling optics 160 for producing a beam of the desired shape in the sample 152, may be present between the output of the wavelength multiplexer 150 and the sample 152. After interacting with the sample in the sample 152, the light may be detected by the photodetector 112. In FIG. 1C, the photodetector 112 is illustrated as being on the opposite side of the sample 152 from the source of the probe light for ease of illustration; in some embodiments the photodetector 112 is positioned on the same side of the sample as the source of the probe light, and the probe light may reach the photodetector 112 after scattering one or more times within the sample. This type of optical path may be important for measurements made by illuminating a first location on the skin of the subject with probe light (transmitted through a transmitting window), and detecting light returning from the skin at a second location near the first location (through a receiving window).

The photodiode signal may be amplified by a suitable amplifier, and converted to a digital signal by an analog to digital converter, and the resulting digital signal may be fed to the controller 155 for further processing. A power meter 170 and a wavelength meter 175 may measure the optical power and wavelength, respectively, of the probe light, and (i) corrections may be made (e.g., by the controller 155) by adjusting, e.g., the drive currents of the lasers or drive currents of heaters controlling the temperatures of respective gratings of the lasers, or (ii) errors in the transmitted power or wavelength may be compensated for when the data are analyzed. The ratio, as a function of wavelength, of (i) the optical power detected by the photodetector 112 to (ii) the optical power transmitted in the probe light may be referred to herein as a “spectrum”.

Estimates of concentrations of biomarkers (e.g., compounds such as glucose, creatinine, urea, lactate, water or alcohol within the tissues of the subject) may be generated, for example, by fitting a measured spectrum with a combination of signatures, each signature being the spectrum that would be expected if a single biomarker were present in the sample at a certain reference concentration.

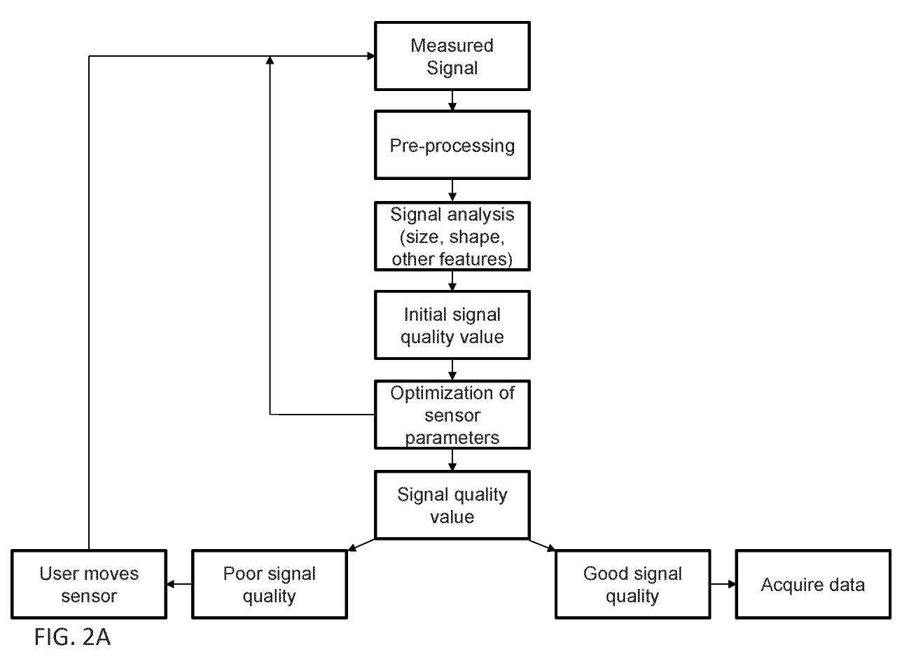

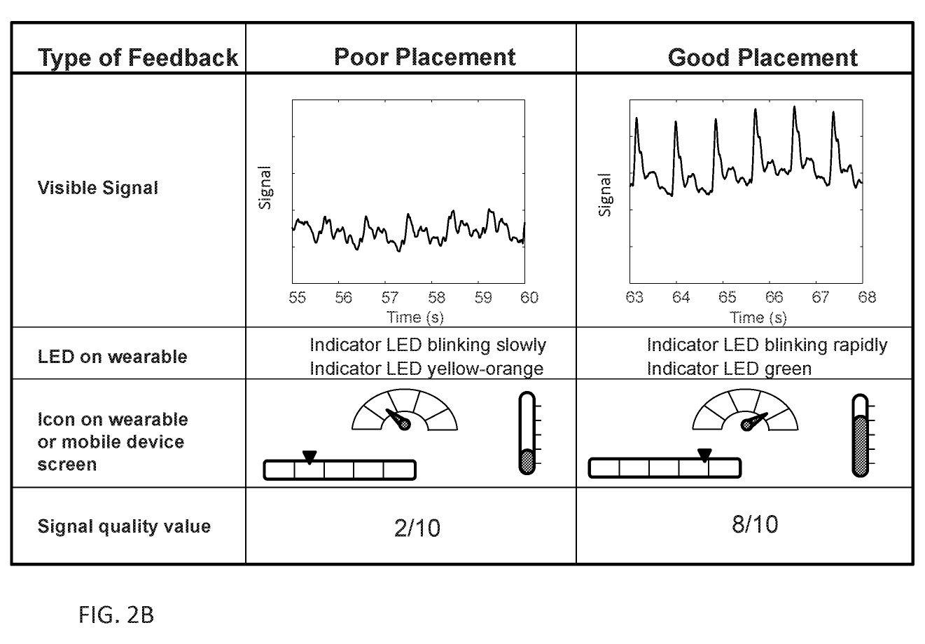

Some embodiments also include a signal quality indicator that the user may refer to in order to locate the optimal position for the second wearable instrument 110. The process of adjusting the second wearable instrument 110 may proceed as illustrated in FIG. 2A, in which the second wearable instrument 110 generates a signal, which is analyzed by one or more processing circuits (e.g., by a processing circuit of the second wearable instrument 110, or by processing circuit of the first wearable instrument 105, or by processing circuit of a portable device (e.g., a mobile telephone) receiving signals from one of the wearable instruments), to generate a signal quality value, which may be displayed to the user (e.g., to the subject, or to a clinician adjusting the position of the second wearable instrument 110) using, e.g., one of the display techniques illustrated in FIG. 2B (illustrated for poor placement and for good placement, of the second wearable instrument 110, in the second and third columns, respectively, of FIG. 2B). Such display techniques may include, e.g., a visual (e.g., a graph) of the signals of interest, an indicator LED that changes blink rate or color, or an iconographical or numerical score displayed on the screen of the wearable or on a connected mobile device. If the signal quality is poor (e.g., if the signal quality indicator is less than a threshold), the user may move the second wearable instrument 110; if the signal quality is good (e.g., if the signal quality indicator is greater than the threshold), the user may leave the second wearable instrument 110 in place (or secure it in place using a clasp).

Signal quality indicators may be calculated in various ways, based on a waveform measured by the second wearable instrument 110. Such a waveform may be represented by a sequence of measurements. For example, for signals influenced by arterial blood flow (e.g., photoplethysmography or speckleplethysmography signals), a signal quality indicator may be a measure of the extent to which the signal exhibits characteristics of the cardiac cycle. For example, frequency-domain analysis of signal power within the first six pulse rate harmonics may be performed, and compared to the signal power (i) at frequencies between the first six pulse rate harmonics or (ii) at frequencies higher than the sixth pulse rate harmonic. For example, the signal quality indicator may be calculated as the ratio of (i) the total power in the first six pulse rate harmonics to (ii) the total power in the signal. As another example, fiducial points corresponding to features of the cardiac cycle (e.g., the S1 and S2 heart sounds) may be identified in the signal and the consistency of their relative timing may be used to calculate a signal quality indicator (e.g., over a number of cycles, the signal quality indicator may be the fraction of the number of fiducial points falling within 15%, of the period of the cardiac cycle, of the time at which they were expected to occur). In other examples, a morphology-based classifier may be employed, in which the signal quality indicator is a measure of the goodness of fit of the signal to a nominal or ideal signal. Some embodiments further include the ability to calibrate the first wearable instrument 105 or the second wearable instrument 110 based on feedback from the signal quality indicator and other measured parameters that may change as a result of repositioning the sensor. For example, it may be that the sensitivity of a first sensor in the second wearable instrument 110 is proportional to a signal quality indicator (derived from the first sensor or from a second sensor); in such a case the first sensor may be calibrated based on the signal quality indicator.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y%” of a first number, it means that the second number is at least (1-Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

Spatially relative terms, such as “beneath”, “below”, “lower”, “under”, “above”, “upper” and the like, may be used herein for ease of description to describe one element or feature’s relationship to another element(s) or feature(s) as illustrated in the figures. It will be understood that such spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

Any numerical range recited herein is intended to include all sub-ranges of the same numerical precision subsumed within the recited range. For example, a range of "1.0 to 10.0" or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1 – 35/100) times 10) and the recited maximum value of 13.5 (i.e., (1 + 35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

Although exemplary embodiments of a repositionable volar module have been specifically described and illustrated herein, many modifications and variations will be apparent to those skilled in the art. Accordingly, it is to be understood that a repositionable volar module constructed according to principles of this disclosure may be embodied other than as specifically described herein. The invention is also defined in the following claims, and equivalents thereof.

What is claimed is:

1. A wearable volar module, comprising:
a biometric sensor configured to produce a photoplethysmography signal and a speckleplethysmography signal,
the volar module being configured to be attached to a wearable strap, and
the wearable strap being configured to be attached to a dorsal module.

2. The wearable volar module of claim 1, wherein the volar module is adjustably positioned along the wearable strap.

3. The wearable volar module of claim 1, wherein the volar module is configured to transmit a signal.

4. The wearable volar module of claim 3, wherein the volar module is configured to transmit the signal to a separate module via a conductive connection.

5. The wearable volar module of claim 4, wherein the volar module is configured to receive power from the separate module via a conductive connection.

6. The wearable volar module of claim 3, wherein the signal is produced by the biometric sensor.

7. The wearable volar module of claim 1, wherein the volar module is configured to analyze a signal produced by the biometric sensor to generate a signal quality value.

8. The wearable volar module of claim 7, wherein the volar module is configured to display an indication of the signal quality value.

9. The wearable volar module of claim 1, wherein the volar module does not comprise a battery, a radio, or an antenna.

10. A system, comprising:
the wearable volar module of claim 1; and
the strap,
the strap being sized and dimensioned to be disposed about a wrist, and
the volar module being attached to the strap.

11. The system of claim 10, further comprising a dorsal module attached to the strap,
wherein the system is configured for the volar module to be capable of being secured to the strap at a first position relative to the dorsal module and at a second position relative to the dorsal module.

12. The system of claim 11, further comprising a conductive connection between the volar module and the dorsal module.

13. The system of claim 12, wherein the volar module is configured:
to transmit a signal, produced by the biometric sensor, to the dorsal module via the conductive connection, and
to receive power from the dorsal module via the conductive connection.

14. The system of claim 13, wherein the dorsal module comprises a radio configured to transmit the signal.

15. The system of claim 13, wherein the dorsal module is configured to transmit a signal to the volar module via the conductive connection.

16. The system of claim 11, wherein the dorsal module comprises a battery configured to supply power to the volar module.

17. The system of claim 11, wherein the system is capable of securing the volar module at a plurality of positions along the strap relative to the dorsal module.

18. The system of claim 17, wherein the plurality of positions span at least 5 mm along the strap.

19. The system of claim 10, wherein the system comprises one or more processing circuits configured to analyze measurement data obtained by the biometric sensor to generate a signal quality value based on the measurement data obtained by the biometric sensor.

20. The system of claim 10, wherein the system is configured to wirelessly transmit measurement data obtained by the biometric sensor.
